(19) United States
(12) Patent Application Publication
(10) Pub. No.: US 2025/0114347 A1
(43) Pub. Date: Apr. 10, 2025

(54) TOPICAL NALTREXONE AS A TREATMENT FOR DRY EYE

(71) Applicant: The Penn State Research Foundation, University Park, PA (US)

(72) Inventors: Patricia J. McLaughlin, University Park, PA (US); Joseph W. Sassani, University Park, PA (US)

(21) Appl. No.: 18/728,779

(22) PCT Filed: Jan. 11, 2023
(86) PCT No.: PCT/US2023/010591

§ 371 Date: Jul. 12, 2024

Related U.S. Application Data
(60) Provisional application No. 63/298,870, filed on Jan. 12, 2022.

(51) Int. Cl. A61K 31/485; A61K 9/00; A61P 27/04

(52) U.S. Cl. CPC A61K 31/485; A61K 9/0048; A61P 27/04

(57) ABSTRACT

Disclosed are methods and compositions for the treatment of dry eye. In particular, the invention relates to methods and compositions for treating dry eye based on topical application of opioid antagonists such as naltrexone.

### CROSS-REFERENCE TO RELATED APPLICATION

This application claims the benefit of U.S. Provisional Application Ser. No. 63/298,870 titled “Topical Naltrexone As A Treatment For Dry Eye” filed Jan. 12, 2022, which is hereby incorporated herein by reference in its entirety.

### STATEMENT REGARDING FEDERALLY SPONSORED RESEARCH OR DEVELOPMENT

This invention was made with government support under Grant No. EY016666 awarded by the National Institutes of Health. The Government has certain rights in the invention.

### BACKGROUND AND SUMMARY

Dry eye is a disorder of the tear film due to tear deficiency or excessive tear evaporation which causes damage to the interpalpebral ocular surface associated with symptoms of ocular discomfort (Smith, The Ocular Surface 5(2): 93-407 (2007)). Chronic dryness leads to pain and irritation that is often debilitating to the subject, preventing the performance of normal daily activities such as reading, driving, among other things. Dry eye is increasing in prevalence as population ages. Approximately 4.9 million Americans 50 years and older have dry eye, and many more have less severe symptoms notable only during contact with adverse contributing factors such as low humidity or contact lens wear (Smith, 2007), as well as the 21 million individuals with diabetes. The number of women affected with dry eye appears to exceed that of men.

Currently dry eye includes two major classes: aqueous tear-deficient dry eye (ADDE) and evaporative dry eye (EDE). ADDE mainly refers to a failure of reflex lacrimal secretion, but also includes a failure of non-reflex water secretion by the conjunctiva. ADDE has two major subclasses, Sjogren Syndrome dry eye (SSDE) and non-SS dry eye. EDE may be intrinsic, where dry eye is due to intrinsic disease affecting lid structures or dynamics, or extrinsic, where ocular surface disease occurs due to some extrinsic exposure such as topical drug preservatives, contact lens wear or vitamin A deficiency. See review by Lemp, The Ocular Surface 5(2): 75-92 (2007).

Substantiated risk factors for developing dry eye include female sex, older age, postmenopausal estrogen therapy, diabetes mellitus, a diet that is low in omega 3 essential fatty acids or has a high ratio of omega 6 to omega 3 fatty acids, refractive surgery, vitamin A deficiency, radiation therapy, bone marrow transplant, hepatitis C, certain classes of systemic and ocular medications including anti-histamines (Smith, 2007). Other risk factors may include HIV, human T cell lymphotropic virus-1 infection, connective tissue diseases, systemic cancer chemotherapy, and certain other medications (Smith, 2007).

Current therapies for dry eye include tear supplementation (e.g., lubricants), tear retention, tear stimulation, tear substitutes, anti-inflammatory therapy, and essentially fatty acids, as discussed in a review article (Pflugfelder, The Ocular Surface 5(2): 163-178, 2007). Over-the-counter lubricants are most frequently prescribed by vision specialists; however lubricants offer only temporary relief, can be expensive, and need to be taken for life. The cause of dry eye is not treated with lubricants.

### SUMMARY OF THE INVENTION

In one aspect, the disclosure provides a composition for treating dry eye comprising 0.5%-3.0% weight/volume of naltrexone, naloxone, or a pharmaceutically acceptable salt thereof, and a pharmaceutically acceptable carrier.

In another embodiment, the disclosure provides a method of treating dry eye in a subject in need thereof, comprising administering topically to the eye or eyes of said subject a composition comprising 0.5%-3% naltrexone, naloxone, or a pharmaceutically acceptable salt thereof and a pharmaceutically acceptable carrier.

### DETAILED DESCRIPTION

Opioid antagonists, such as naltrexone and naloxone, are known as antagonists of an opioid receptor and are used primarily in the management of dependence on drugs (e.g., opioids), alcohol, and nicotine. The IUPAC name for naltrexone is 17-(cyclopropylmethyl)-4,5α-epoxy-3,14-dihydroxymorphinan-6-one. Naltrexone can be described as a substituted oxymorphone, where the tertiary amine methyl-substituent is replaced with methylcyclopropane. Naloxone is also known as dihydroxymorphinan-6-one.

Without being bound by any particular theory or mechanism, the inventors believe that elevated levels of opioids in dry eye patients depress neurotransmission in nerves regulating secretion of tears. Naltrexone interrupts the depressive effect exerted by opioid-opioid receptor interactions, thereby releasing glands to secrete tears. In addition, the excess of some opioids (Met-enkephalin) in at least some individuals suffering from dry eye act as growth factors (termed opioid growth factor) and depress cell proliferation of glandular cells. Naltrexone interrupts the interfacing of opioids from classical opioid receptors, thereby blocking the inhibitory action of this opioid system on neurotransmission, as well as blocking the opioid growth factor from its non-classical opioid receptor, opioid growth factor receptor, and stimulating cell proliferation of secretory cells.

The inventors disclose herein the surprising discovery that there is a narrow range of effective concentrations of naltrexone (NTX) for the treatment of dry eye. Surprisingly, high concentrations of NTX actually cause or exacerbate dry eye. Thus, the instant disclosure represents a valuable contribution to the treatment of dry eye using opioid antagonists that would not have been obvious to one of skill in the art.

### Compositions for the Treatment of Dry Eye

In one aspect of the current disclosure, compositions for treating dry eye are provided. In some embodiments, the compositions comprise 0.5%-3.0% weight/volume of naltrexone, naloxone, or a pharmaceutically acceptable salt thereof, and a pharmaceutically acceptable carrier. In some embodiments, the effective amount is about 0.5%-2% weight/volume. In some embodiments, the effective amount is about 0.5%-1.5% weight/volume.

The formulation of the topical opioid antagonist solution comprising an opioid antagonist is clearly needed for function. Thus, in some embodiments, the compositions further comprise 0.5% carboxyl methyl cellulose sodium, sodium borate, 0.75% sodium chloride, 0.001% polidronium chloride weight/volume, and wherein the composition has a pH of about 7.2. In some embodiments, the compositions comprise boric acid.

### Methods for Treating Dry Eye

The disclosed compositions have been experimentally demonstrated to effectively reduce dry eye in vivo. Accordingly, in another aspect of the current disclosure methods of treating dry eye in a subject in need thereof are provided. In some embodiments, the methods comprise administering topically to the eye or eyes of said subject a composition comprising 0.5%-3% naltrexone, naloxone, or a pharmaceutically acceptable salt thereof and a pharmaceutically acceptable carrier. In some embodiments, the effective amount is about 0.5%-2% weight/volume. In some embodiments, the effective amount is about 0.5%-1.5% weight/volume. In some embodiments, the composition further comprises 0.5% carboxyl methyl cellulose sodium, sodium borate, 0.75% sodium chloride, 0.001% polidronium chloride, and wherein the composition has a pH of 7.2. In some embodiments, the composition comprises boric acid. In some situations repeated administration of the disclosed compositions may be useful in the treatment of dry eye. Therefore, in some embodiments, the administration takes place once per day. In some embodiments, the administration takes place twice per day.

Several tests for determining the extent of dry eye in a subject are known in the art. An exemplary test for determining the presence or level of dry eye in a subject is a Schirmer test. As used herein, “Schirmer's test” refers to a test which measures both basal and reflex tear production. In this test, the end of the appropriately bent test strip is placed inside the lower eyelid at the lid margin without anesthesia, after 5 minutes, the strip is removed, and the amount of wetting, or tear fluid volume, is measured in millimeters. In general, any value below 10 mm is considered abnormal. Thus, Schirmer's test represents a valuable way to measure the efficacy of the disclosed compositions for the treatment of dry eye. Accordingly, in some embodiments, the method results in normal Schirmer's test results after 5 days of administration. In some embodiments, the method results in normal Schirmer's test results after 1 day of administration. In some embodiments, the method results in the subject having a tear fluid volume greater than about 10 mm of filter paper in about 5 minutes.

In accordance with the present invention, the therapeutic method of treating dry eye involves identifying a subject suffering from dry eye symptoms and administering to the eyes of such subject an effective amount of naltrexone or other opioid antagonists.

Subjects contemplated by the present invention include any mammalian subjects, particularly human subjects, dogs, cats and horses. Although dry eye may occur in diabetic patients, a large population of normal, non-diabetic individuals also suffer from dry eye. The composition and method of the present invention can treat both diabetic and non-diabetic subjects.

Dry eye symptoms can include dryness, sandy feeling, burning, redness, crusting on lashes, itchiness not related to allergy, stickiness, and eyes stuck shut in the morning.

By “treating dry eye” it is meant to include complete or partial alleviation of all or some of symptoms of dry eye, and/or prevention or inhibition of the symptoms of dry eye. The treatment includes, but is not limited to, for example, promoting basal lacrimal secretion.

The opioid antagonist-containing compositions of the present invention can include other active agents for treatment of dry eye, including, but not limiting to, anti-infective agents, antibiotics, antiviral agents, anti-inflammatory drugs, anti-allergic agents including anti-histamines, vasoconstrictors, vasodilators, local anesthetics, analgesics, intraocular pressure-lowering agents, immunoregulators, anti-oxidants, vitamins and minerals, proteases and peptidases that breakdown endogenous opioids, and the like.

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject in need of treatment for dry eye. A subject in need thereof may include a subject suffering from dry eye, or a subject with dry eye characterized by aberrant or hyperactive opioid receptor signaling in the eye.

The term “subject” may be used interchangeably with the terms “individual” and “patient” and includes human and non-human mammalian subjects.

The disclosed compounds, pharmaceutical compositions, and methods may be utilized to treat diseases and disorders associated with opioid receptor signaling activity and/or expression which may include, but are not limited to dry eye.

The term “alkyl” as used herein refers to a saturated straight or branched hydrocarbon, such as a straight or branched group of 1-12, 1-10, or 1-6 carbon atoms, referred to herein as C₁-C₁₂ alkyl, C₁-C₁₀-alkyl, and C₁-C₆-alkyl, respectively.

The term “alkylene” refers to a diradical of an alkyl group. An exemplary alkylene group is —CH₂CH₂—.

The term “haloalkyl” refers to an alkyl group that is substituted with at least one halogen, for example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂-alkenyl, C₂-C₁₀-alkenyl, and C₂-C₆-alkenyl, respectively. A “cycloalkene” is a compound having a ring structure (e.g., of 3 or more carbon atoms) and comprising at least one double bond.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂-alkynyl, C₂-C₁₀-alkynyl, and C₂-C₆-alkynyl, respectively.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈-cycloalkyl,” derived from a cycloalkane. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkyl group is not substituted, i.e., it is unsubstituted.

The term “cycloalkylene” refers to a diradical of a cycloalkyl group.

The term “partially unsaturated carbocyclyl” refers to a monovalent cyclic hydrocarbon that contains at least one double bond between ring atoms where at least one ring of the carbocyclyl is not aromatic. The partially unsaturated carbocyclyl may be characterized according to the number or ring carbon atoms. For example, the partially unsaturated carbocyclyl may contain 5-14, 5-12, 5-8, or 5-6 ring carbon atoms, and accordingly be referred to as a 5-14, 5-12, 5-8, or 5-6 membered partially unsaturated carbocyclyl, respectively. The partially unsaturated carbocyclyl may be in the form of a monocyclic carbocycle, bicyclic carbocycle, tricyclic carbocycle, bridged carbocycle, spirocyclic carbocycle, or other carbocyclic ring system. Exemplary partially unsaturated carbocyclyl groups include cycloalkenyl groups and bicyclic carbocyclyl groups that are partially unsaturated. Unless specified otherwise, partially unsaturated carbocyclyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the partially unsaturated carbocyclyl is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The terms “heterocyclyl” and “heterocyclic group” are art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using 5 Cx-Cx nomenclature where x is an integer specifying the number of ring atoms. For example, a C₃-C₇ heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position.

The terms “amine” and “amino” are art-recognized and refer to both unsubstituted and substituted amines, wherein substituents may include, for example, alkyl, cycloalkyl, heterocyclyl, alkenyl, and aryl.

The terms “alkoxyl” or “alkoxy” are art-recognized and refer to an alkyl group, as defined above, having an oxygen radical attached thereto. Representative alkoxyl groups include methoxy, ethoxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and the like.

The term “carbonyl” as used herein refers to the radical —C(O)—.

The term “carboxy” or “carboxyl” as used herein refers to the radical —COOH or its corresponding salts, e.g. —COONa, etc.

The term “amide” or “amido” or “carboxamido” as used herein refers to a radical of the form —R¹C(O)N(R³)—, —R¹C(O)N(R²)R³—, —C(O)NR²R³, or —C(O)NH₂, wherein R¹, R² and R³ are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereo isomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

### Pharmaceutical Compositions

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form which is pharmaceutically acceptable; illustratively, they can be orally administered pharmaceutical compositions. Such pharmaceutical compositions contain an s of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures.

The compounds for use according to the methods of disclosed herein may be administered as a single compound or a combination of compounds.

As indicated above, pharmaceutically acceptable salts of the compounds are contemplated and also may be utilized in the disclosed methods. The term “pharmaceutically acceptable salt” as used herein, refers to salts of the compounds, which are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

Acids commonly employed to form acid addition salts may include inorganic acids such as hydrochloric acid, hydrobromic acid, hydroiodic acid, sulfuric acid, phosphoric acid, and the like, and organic acids such as p-toluenesulfonic, methanesulfonic acid, oxalic acid, p-bromophenylsulfonic acid, carbonic acid, succinic acid, citric acid, benzoic acid, acetic acid, and the like. Examples of suitable pharmaceutically acceptable salts may include the sulfate, pyrosulfate, bisulfate, sulfite, bisulfate, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleat-, butyne-.1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, naphthalene-2-sulfonate, mandelate, and the like.

Base addition salts include those derived from inorganic bases, such as ammonium or alkali or alkaline earth metal hydroxides, carbonates, bicarbonates, and the like. Bases useful in preparing such salts include sodium hydroxide, potassium hydroxide, ammonium hydroxide, potassium carbonate, sodium carbonate, sodium bicarbonate, potassium bicarbonate, calcium hydroxide, calcium carbonate, and the like.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

The pharmaceutical compositions may be utilized in methods of treating a disease or disorder associated with the biological activity of ubiquitin specific peptidase 22 (USP22). As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for treating a disease or disorder associated with dry eye.

By “treating dry eye” it is meant to include complete or partial alleviation of all or some of symptoms of dry eye, and/or prevention or inhibition of the symptoms of dry eye. The treatment includes, but is not limited to, for example, promoting basal lacrimal secretion.

As used herein, a “drop” of a solution refers to about 38 μl of said solution. In some embodiments, a drop of a solution is about 50 μl of said solution. In some embodiments, a drop of a solution is about 25-75 μl of said solution.

A composition containing an opioid antagonist such as naltrexone or naloxone can be administered topically to the eye or eyes of a subject suffering from dry eye. Topical administration includes directly applying, laying, or spreading on or around the eye, e.g., by use of an applicator such as a wipe, a contact lens, a dropper, or a spray.

A composition containing an opioid antagonist is formulated for convenient topical administration. Forms of the composition include, but are not limited to, solutions, ointments, gels, emulsions, suspensions, gel shields, and the like.

In one embodiment, naltrexone or another opioid antagonist is contained in an aqueous-based cream excipient, which can be applied to the eye at bedtime, but may also be applied any time throughout the day.

In another embodiment, a composition containing an opioid antagonist such as naltrexone or naloxone is formulated as a solution or suspension and is applied topically in the form of eye drops. Any solution suitable for topical application in which an opioid antagonist (such as naltrexone or naloxone) is soluble can be used; e.g., sterile water, Sorenson's phosphate buffer. In one embodiment, the composition can be incorporated in eye drop solutions for contact lens, washing solutions for contact lens, or preserving solutions for contact lens.

In other embodiments, a composition containing an opioid antagonist is made to have properties such as sustained-release or improved stability. For example, a polymeric matrix composition containing naltrexone can be topically applied to the eye to achieve sustained release.

Compositions containing an opioid antagonist can include additional ingredients, additives or carrier suitable for use in contact on or around the eye without undue toxicity, incompatibility, instability, irritation, allergic response, and the like. Additives such as solvents, bases, solution adjuvants, suspending agents, thickening agents, emulsifying agents, stabilizing agents, buffering agents, isotonicity adjusting agents, soothing agents, preservatives, corrigents, flavoring agents, coloring agents, excipients, binding agents, lubricants, surfactants, absorption-promoting agents, dispersing agents, preservatives, solubilizing agents, and the like, can be added to a formulation where appropriate.

The opioid antagonist-containing compositions of the present invention can include other active agents for treatment of dry eye, including, but not limiting to, anti-infective agents, antibiotics, antiviral agents, anti-inflammatory drugs, anti-allergic agents including anti-histamines, vasoconstrictors, vasodilators, local anesthetics, analgesics, intraocular pressure-lowering agents, immunoregulators, anti-oxidants, vitamins and minerals, proteases and peptidases that breakdown endogenous opioids, and the like. A typical daily dose may contain from about 0.01 mg/kg to about 100 mg/kg (such as from about 0.05 mg/kg to about 50 mg/kg and/or from about 0.1 mg/kg to about 25 mg/kg) of each compound used in the present method of treatment.

Compositions can be formulated in a unit dosage form, each dosage containing from about 1 to about 500 mg of each compound individually or in a single unit dosage form, such as from about 5 to about 300 mg, from about 10 to about 100 mg, and/or about 25 mg. The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

An opioid antagonist-containing composition of the present invention is being administered to a subject in need thereof at an effective amount to treat the dry eye condition. The effective amount of the composition for a particular individual can depend on the severity of the condition of the individual, the type of formulation being applied, the frequency of administration, and the duration of the treatment. Suitably, the formulation is administered via drops to the eye.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Opioid Antagonists and Uses Thereof for Treatment of Dry Eye

Disclosed are compounds, pharmaceutical compositions comprising the compounds, and methods of using the compounds and pharmaceutical compositions for treating a subject having or at risk for developing dry eye. The disclosed compounds may inhibit the biological activity of opioid receptors. As such, the disclosed compounds and pharmaceutical compositions may be utilized in methods for treating a subject having or at risk for developing a disease or disorder that is associated with opioid receptor signaling activity in the eye which may cause dry eye in a subject.

In some embodiments, a subject is treated topically with an opioid receptor antagonist daily. In some embodiments, a subject is treated topically with an opioid receptor antagonist twice daily. In some embodiments, a subject is treated topically for at least a week with opioid receptor antagonists. In some embodiments, a subject is treated topically for at least a month with opioid receptor antagonists.

In some embodiments, the compositions comprising an opioid receptor antagonist has a concentration of about 0.5% to 3% opioid receptor antagonist weight/volume. In some embodiments, the compositions comprising an opioid receptor antagonist has a concentration of about 0.5% to 2% opioid receptor antagonist weight/volume. In some embodiments, the compositions comprising an opioid receptor antagonist has a concentration of about 0.5% to 1.5% opioid receptor antagonist weight/volume. In some embodiments, the compositions comprise about 0.5% carboxyl methyl cellulose sodium, sodium borate, about 0.75% sodium chloride, about 0.001% polidronium chloride weight/volume, and wherein the composition has a pH of about 7.2. In some embodiments, the compositions comprise boric acid.

In some embodiments, the compositions and methods of the current disclosure are effective at treating dry eye in both male and female subjects.

In some embodiments, treatment of a subject with the compositions and methods of the current disclosure increases tear fluid volume to a normal level after 1 treatment. In some embodiments, treatment of a subject with the compositions and methods of the current disclosure increases tear fluid volume to a normal level after 2, 3, 4, 5, 6, 7, 8, 9, or 10 treatments. In some embodiments, treatment of a subject in need thereof with the compositions and methods of the current disclosure continues to provide relief from dry eye in the subject 1 day after cessation of treatment. In some embodiments, treatment of a subject in need thereof with the compositions and methods of the current disclosure continues to provide relief from dry eye in the subject 2 days after cessation of treatment. In some embodiments, treatment of a subject in need thereof with the compositions and methods of the current disclosure continues to provide relief from dry eye in the subject 3 days after cessation of treatment.

Exemplary opioid receptor antagonists for use in the disclosed compositions may include, but are not limited to, a compound having a formula selected from the group consisting of:

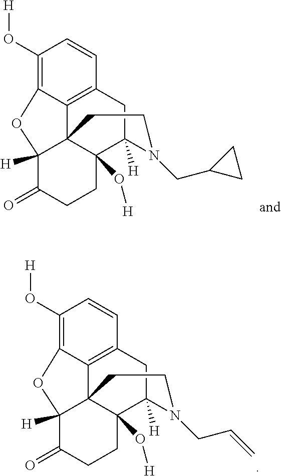 [SMILES: O=C1CC[C@@]2(O)[C@H]3Cc4ccc(O)c5c4[C@@]2(CCN3CC2CC2)[C@H]1O5 ; C=CCN1CC[C@]23c4c5ccc(O)c4O[C@H]2C(=O)CC[C@@]3(O)[C@H]1C5]

In some embodiments of the disclosed compositions, the compositions comprise: N-Cyclopropylmethylnoroxymorphone and n-Allylnoroxymorphone.

In another aspect of the current disclosure, methods of treating dry eye in a subject in need thereof are provided. In some embodiments, the methods comprise administering topically to the eye or eyes of said subject a composition comprising 0.5%-3% naltrexone, naloxone, or a pharmaceutically acceptable salt thereof and a pharmaceutically acceptable carrier.

Exemplary therapeutic agents for use in the disclosed methods may include, but are not limited to, a compound having a formula selected from the group consisting of:

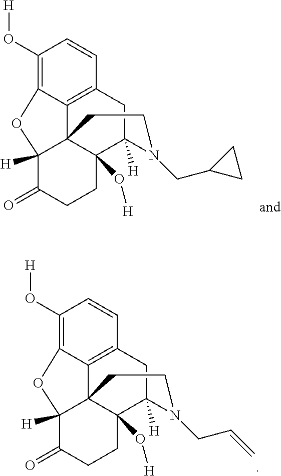 [SMILES: O=C1CC[C@@]2(O)[C@H]3Cc4ccc(O)c5c4[C@@]2(CCN3CC2CC2)[C@H]1O5 ; C=CCN1CC[C@]23c4c5ccc(O)c4O[C@H]2C(=O)CC[C@@]3(O)[C@H]1C5]

In some embodiments of the disclosed methods, the subject is administered a compound selected from the group consisting of: N-Cyclopropylmethylnoroxymorphone and n-Allylnoroxymorphone.

### EXAMPLES

The following Examples are illustrative and should not be interpreted to limit the scope of the claimed subject matter.

### Example 1 Formulations of Naltrexone for Reducing Dry Eye Have A Narrow Effective Range

Four solutions were prepared with 0%, 0.5%, 1.5%, and 3% naltrexone (NTX). Previous formulations contained 0.003% NTX.

Adult male and female Sprague-Dawley rats were rendered hyperglycemic for 6-8 weeks, with blood glucose levels >600 mg/dL. Age at time of testing was 15-18 weeks of age.

Experimental paradigm: 1-4 rats were tested with each dose (mixture of male and females). Left eyes only were treated twice daily with one drop.
Schirmer test for baseline (to determine tear production and establish dry eye)
Add drop at 0800 and again at 1700 hr (4:40-6:00 pm)
Take Schirmer test 24 hr later
Add drop at 0800 and again at 1700 hr (4th drop)
Take Schirmer test 48 hr after first drop

### Results:

3% NTX caused dry eye in a normal rat and did not reverse dry eye in the diabetic rats (both male and female). In contrast, 1.5% and 0.5% NTX reversed dry eye while 1.5% was most effective. As expected, vehicle control (0% NTX) had no effect. Interestingly, the highest dose (3% NTX) left at RT for 3 weeks allowing some decomposition of the NTX was effective at reversing dry eye, thus further demonstrating that a function of naltrexone decaying to dosages less than 3% are effective.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A composition for treating dry eye comprising from about 0.5% to about 3.0% weight/volume of naltrexone, naloxone, or a pharmaceutically acceptable salt thereof, and a pharmaceutically acceptable carrier.

2. The composition of claim 1, wherein the effective amount is from about 0.5% to about 2% weight/volume.

3. The composition of claim 1, wherein the effective amount is from about 0.5% to about 1.5% weight/volume.

4. The composition of claim 1, wherein further comprising about 0.5% carboxyl methyl cellulose sodium, sodium borate, about 0.75% sodium chloride, about 0.001% polidronium chloride weight/volume, and wherein the composition has a pH of about 7.2.

5. The composition of claim 1, wherein the composition comprises boric acid.

6. A method of treating dry eye in a subject in need thereof, comprising administering topically to the eye or eyes of said subject a composition comprising from about 0.5% to about 3% naltrexone, naloxone, or a pharmaceutically acceptable salt thereof and a pharmaceutically acceptable carrier.

7. The method of claim 6, wherein the effective amount is from about 0.5% to about 2% weight/volume.

8. The method of claim 6, wherein the effective amount is from about 0.5% to about 1.5% weight/volume.

9. The method of claim 6, wherein the composition further comprises about 0.5% carboxyl methyl cellulose sodium, sodium borate, about 0.75% sodium chloride, about 0.001% polidronium chloride, and wherein the composition has a pH of about 7.2.

10. The method of claim 6, wherein the composition comprises boric acid.

11. The method of claim 6, wherein the administration takes place once per day.

12. The method of claim 6, wherein the administration takes place twice per day.

13. The method of claim 6, wherein the method results in normal Schirmer's test results after 5 days of administration.

14. The method of claim 6, wherein the method results in normal Schirmer's test results after 1 day of administration.

15. The method of claim 6, wherein the method results in a tear fluid volume greater than about 10 mm of filter paper in about 5 minutes.
